(19) United States
(12) Patent Application Publication
(10) Pub. No.: US 2007/0141662 A1
(43) Pub. Date: Jun. 21, 2007

(54) Fusion proteins and methods of cleavage of such proteins

(75) Inventors: Henning Ralf Stennicke, Kokkedal (DK); Lars Fogh Iversen, Holte (DK); Guy S. Salvesen, Encinitas, CA (US)

Correspondence Address: NOVO NORDISK, INC.;PATENT DEPARTMENT, 100 COLLEGE ROAD WEST, PRINCETON, NJ, 08540, US

(73) Assignees: Novo Nordisk A/S, Bagsvaerd (DK); The Burnham Institute, La Jolla, CA (US)

(21) Appl. No.: 11/439,896

(22) Filed: May 24, 2006

Related U.S. Application Data
(63) Continuation of application No. PCT/DK04/00815, filed on Nov. 24, 2004.

(30) Foreign Application Priority Data
Nov. 24, 2003 (DK) PA 2003 01729

(51) Int. Cl. C12P 21/06; A61K 38/28; C07K 14/745; C07K 14/56

(52) U.S. Cl. 435/68.1; 530/303; 530/351; 530/383

(57) ABSTRACT

The invention provides a method of processing recombinant proteins by using aspartate specific proteases.

### CROSS-REFERENCE TO RELATED PATENT APPLICATIONS

This patent application is a continuation of copending International Patent Application PCT/DK2004/000815, filed Nov. 24, 2004 (published as WO 2005/049847), which designates the US, and claims the benefit of Danish Patent Application PA200301729, filed Nov. 24, 2003, and U.S. Provisional Patent Applications 60/526,021, filed Dec. 1, 2003, and 60/590,842, filed Jul. 23, 2004, the entirety of each of which is hereby incorporated by reference.

### FIELD OF THE INVENTION

The invention provides the construction of fusion proteins and cleavage of such fusion proteins in a specific manner by using caspases.

### BACKGROUND OF THE INVENTION

Recombinant production of peptides and proteins sometimes requires fusion of peptide or proteins in order to achieve certain properties of the desired protein. The present invention provides a method of cleaving such fusion proteins and suitable cleavage sites.

Aspartate specific proteases are known in the art. Among these the caspases and the caspase activator Granzyme B are among the best described. These enzymes are all among the most specific proteolytic enzymes known to date due to their extended substrate recognition site. The caspases are cystein dependent aspartate specific proteases, which are well known for the ability to act as highly specific restriction proteases in vivo where they cleave a small subset of proteins in order to promote the development of the morphology characteristic of cells undergoing programmed cell death or apoptosis. Different Asp specific proteases are known in the art and described in (Thornberry Chem Biol. 1998; 5:R97-103.; Stennicke & Salvesen Biochim. Biophys. Acta 1998; 1387:17-31). The different proteases have identical primary specificities, but very different secondary preferences and thus allows for the selection of that specific enzyme and matching cleavage site motif which ensures cleavage at the desired site while minimizing cleavage at secondary sites within the protein of interest while ensuring generation of the active in vivo form.

Numerous other fusion protein/processing systems are available today, however, most of these are highly sensitive to reducing conditions. The caspases, being thiol proteases, are on the other hand fully active under reducing conditions and remains active for some time in up to 4 M urea making them ideally suited for removal of tags or fusion partners in unfolded proteins.

### SUMMARY OF THE INVENTION

The invention provides a method of producing a peptide comprising the steps of expression the peptide (e.g. as a fusion protein) which at the amino-terminal of the peptide has a sequence specific for an aspartate specific protease for the cleavage of fusion proteins and contacting the peptide with at least one aspartate-specific protease to liberate the peptide from the starting peptide—(e.g. the fusion protein).

In an embodiment of the invention the asparte specfic protease is selected from caspases 1, 2, 3, 4, 5, 6, 7, 8, 9 or 10, Granzyme B or CED-3

The invention provides the method as described above wherein the fusion protein is expressed in a host cell.

The invention provides the fusion protein between IL-21 and the sequence specific for asparte specific proteases.

The invention provides DNA encoding any of the fusion proteins described.

The invention provides a recombinant vector comprising the DNA encoding any of the proteins described.

The invention also provides a unicellular host organism containing a vector comprising the DNA encoding any of the proteins of above.

### DEFINITIONS

In the context of the present invention IL-21 is defined as the sequence disclosed in WO00/53761 as SEQ ID No.:2. The invention also embraces functional derivatives and fragments thereof.

In the context of this invention “caspase” refers to any caspase regardless of origin or specificity and includes also proteases with caspase-like substrate specificity—such as Granzyme B.

The reference to positions Px and P'x refers to positions x aminoacids to the N-terminal and C-terminal respectively relative to the cleavage site (Schechter & Berger (1967) Biochem. Biophys. Res. Commun. 27, 157).

In the following the one-letter abbreviation for amino acid residues are used.

The terms peptide, polypeptide or protein are used interchangeably herein.

### DESCRIPTION OF THE INVENTION

The protein polypeptides of the present invention, including full-length proteins, protein fragments (e.g. ligand-binding fragments), and fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed.(Cold Spring Harbor Laboratory Press, Cold Spring Harbor, N.Y., 1989), and Ausubel et al., ibid.

It is to be recognized that according to the present invention, when a cDNA is claimed as described above, it is understood that what is claimed are both the sense strand, the anti-sense strand, and the DNA as double-stranded having both the sense and anti-sense strand annealed together by their respective hydrogen bonds. Also claimed is the messenger RNA (mRNA) which encodes the polypeptides of the present invention, and which mRNA is encoded by the above-described cDNA. A messenger RNA (mRNA) will encode a polypeptide using the same codons as those defined above, with the exception that each thymine nucleotide (T) is replaced by a uracil nucleotide (U).

As mentioned, different asp specific proteases are known in the art and described. As a general rule caspases/granzyme B prefer the following motifs:

| Protease | Preference |
|---|---|
| Caspase-2, -3, -7 and CED-3 | DEXaaD-Zaa |
| Caspase-1, -4 and -5 | (Y/F/W)VXaaD-Zaa |
| Caspase-6, -8, -9, -10 and Granzyme B | (I/L/V)EXaaD-Zaa |

Where Xaa and Zaa can be any amino acid residue, with amino acids such as H, T and V being the preferred substituents for Xaa and S, T, G or A being preferred as Zaa, although large aromatic groups also are well tolerated (Stennicke et al. Biochem. J. (2000) 350, 563-568).

In particular for Caspase I the most preferred P4 is Y or F

For Caspase 6 the most preferred P4 is Thr or Val;

For caspase 8 a preferred P4 is Leu:

For the position P1 all caspases prefer Asp.

The position P'1 in caspases are the preferred Gly, Ser, Ala, Phe or Tyr. The most preferred are Gly, Ser or Ala.

Preferred linkers include, but not limited to:

For Caspase 1: YVHD-A, LESD-N, WEHD-A

Caspase 3 and 7: DETD-S, DEVD-G, DMQD-N, DEVD-G and DGPD-G

Caspase 6: VEID-N, VEVD-A

The above linkers demonstrate selectivity in the linking moiety for cleavage by caspases.

In an embodiment of the invention the protease is a caspase 3.

In an embodiment of the invention the peptide is selected from the group consisting of aprotinin, tissue factor pathway inhibitor or other protease inhibitors, insulin or insulin precursors, human or bovine growth hormone, interleukine, glucagon, Glucagon₁₋₃₇ (oxyntomodulin), GLP-1, GLP-2, IGF-I IGF-II, tissue plasminogen activator, transforming growth factor α or β, platelet-derived growth factor, GRF (growth hormone releasing factor), immunoglubolines, EPO, TPA, protein C blood coagulation factors such as FVII, FVIII, FV, FX, FIX or FXIII , exendin-3 or exentidin-4 or functional analogues of any of the above.

In another embodiment of the invention the peptide is an interleukine such as IL-20, IL-21, IL-28, IL-29 or IL-31 and the corresponding receptor molecules and soluble receptor molecules. In a specific embodiment of the invention the peptide is IL-21.

A specific embodiment is a fusion protein as above wherein the construct contains the linker sequence DEXaaD, or (Y/F/M/W)VXaaD, or (I/L/V)EXaaD, wherein Xaa is any aminoacid.

Another specific embodiment is a fusion protein as any of the above wherein the sequence added to the peptide is the sequence DEXaaD and wherein Xaa is any amino acid and the protease is caspase 2,3, 7 or CED-3.

Another specific embodiment is the fusion proteins as any of the above wherein the sequence added to the peptide is the sequence (Y/F/W)VXaaD and wherein Xaa is any amino acid and the protease is caspase 1, 4 or 5.

Another specific embodiment is a fusion protein as any of the above wherein the sequence added to the peptide is the sequence (I/L/V)EXaaD and wherein Xaa is any amino acid and the protease is caspase 6, 8, 9, 10 or Granzyme B.

Another specific embodiment is a fusion protein as any of the above wherein the sequence added is DETD, DEVD, DMQD, DEVD or DGPD and the protease is Caspase 3 or 7:

A specific embodiment of the invention is the recombinant IL-21 sequence in the C-terminal sequence and a construct recognisable by an aspartate specific protease as any of the above.

The above preferred embodiments are not to be construed as limiting to the invention. The selection of the linking moiety is a selection of specificity and kinetic considerations. The caspases has activity towards other motifs too, but with a lower selectivity and for some also a much lower specific activity.

Thus using a linker of this format any protein can be linked to the terminus of another protein to form a fusion protein. In a preferred embodiment this protein in IL-21.

The sequence preceding the protein or peptide of interest may be any sequence suitable for facilitating protein folding and/or purification. These sequences include, but are not limited to, myc-, T7-, HSV- , V5-, HA-, FLAG-, strep-tags, Glutathion transferase (GST), Green Fluorescent Protein (GFP), Thioredoxin (e.g. E. Coli TrxA), His₆ (-HHHHHH-) and variants thereof, chitin binding protein, maltose binding protein.

In a preferred embodiment of the present invention Caspase 3 is used due to its rare recognition site, however, other enzymes may be employed depending on the primary sequence of the protein of interest. This particular enzyme is characterised by a strict preference for Asp in P1 and P4 and Glu in P3 (previously described in Thornberry et al. J Biol Chem. 1997 Jul. 18; 272(29):17907-11.)

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”) DNA Cloning: A Practical Approach, Volumes I and II /D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

In an embodiment of the invention the peptide is aprotinin, tissue factor pathway inhibitor or other protease inhibitors, insulin or insulin precursors, human or bovine growth hormone, interleukin, glucagon, Glucagon₁₋₃₇ (oxyntomodulin), GLP-1, GLP-2, IGF-I, IGF-II, tissue plasminogen activator, transforming growth factor α or β, platelet-derived growth factor, GRF (growth hormone releasing factor), immunoglubolines, EPO, TPA, protein C, blood coagulation factors such as FVII, FVIII, FV, FX, FIX or XIII as well as exendin-3 or exentidin-4, and functional analogues of any of the above. In an embodiment of the invention the peptide is an interleukine such as IL-20, IL-21, IL-28, IL-29 or IL-31 and the corresponding receptor molecules and soluble receptor molecules. In a specific embodiment of the invention the peptide is IL-21.

In the present context, the term “functional analogue” is meant to indicate a protein with a similar function as the native protein. The protein may be structurally similar to the native protein and may be derived from the native protein by addition of one or more amino acids to either or both the C- and N-terminal end of the native protein, substitution of one or more amino acids at one or a number of different sites in the native amino acid sequence, deletion of one or more amino acids at either or both ends of the native protein or at one or several sites in the amino acid sequence, or insertion of one or more amino acids at one or more sites in the native amino acid sequence. Furthermore the protein may be acylated in one or more positions, vide WO 98/08871 which discloses acylation of GLP-1 and analogues thereof and in WO 98/08872 which discloses acylation of GLP-2 and analogues thereof. An example of an acylated GLP-1 derivative is Lys²⁶(N^{ε}-tetradecanoyl)-GLP-1₍₇₋₃₇₎ which is GLP-1₍₇₋₃₇₎ wherein the ε-amino group of the Lys residue in position 26 has been tetradecanoylated.

An insulin analogue is an insulin molecule having one or more mutations, substitutions, deletions and or additions of the A and/or B amino acid chains relative to the human insulin molecule. The insulin analogues are preferably such wherein one or more of the naturally occurring amino acid residues, preferably one, two, or three of them, have been substituted by another codable amino acid residue. Thus position 28 of the B chain may be modified from the natural Pro residue to one of Asp, Lys, or lie. In another embodiment Lys at position B29 is modified to Pro; Also, Asn at position A21 may be modified to Ala, Gln, Glu, Gly, His, lle, Leu, Met, Ser, Thr, Trp, Tyr or Val, in particular to Gly, Ala, Ser, or Thr and preferably to Gly. Furthermore, Asn at position B3 may be modified to Lys. Further examples of insulin analogues are des(B30) human insulin, insulin analogues wherein PheB1 has been deleted; insulin analogues wherein the A-chain and/or the B-chain have an N-terminal extension and insulin analogues wherein the A-chain and/or the B-chain have a C-terminal extension. Thus one or two Arg may be added to position B1, or one Arg to position B30 and one Arg to position B31. Also, precursors or intermediates for other proteins may be treated by the method of the invention. An example of such a precursor is an insulin precursor which comprises the amino acid sequence B(1-29)-AlaAlaLys-A(1-21) wherein A(1-21) is the A chain of human insulin and B(1-29) is the B chain of human insulin in which Thr(B30) is missing. Combinations of the above analogues are also included. Thus the insulin analogue A21GIy- B30Arg-B31Arg is within the insulin analogues of the invention. Finally, the insulin molecule may be acylated in one or more positions, such as in the B29 position of human insulin or desB30 human insulin. Examples of acylated insulins are N^{εB29}-tetradecanoyl GIn^{B3} des(B30) human insulin), N^{εB29}-tridecanoyl human insulin, N^{εB29}-tetradecanoyl human insulin, N^{εB29}-decanoyl human insulin, and N^{εB29}-dodecanoyl human insulin.

“IL-21” is described in International Patent Application publication no. WO 00/53761, published Sept. 14, 2000, which is hereby incorporated in this application in its entirety, discloses IL-21 (as “Zalphal 11 ligand”) as SEQ ID No. 2, which is hereby incorporated in this application in its entirety, as well as methods for producing it and antibodies thereto and a polynucleotide sequence encoding IL-21 as SEQ ID No. 1 in the aforementioned application. The invention comprises their orthologs comprising at least 70%, at least 80%, at least 90%, at least 95%, or greater than 95%. The present invention also includes the use of polypeptides that comprise an amino acid sequence having at least 70%, at least 80%, at least 90%, at least 95% or greater than 95% sequence identity to the sequence of amino acid residues. Methods for determining percent identity are described below. The polypeptides of the present invention have retained all or some of the biological activity of the native counterparts. Some of the polypeptides may also have a biological activity which is higher than the biological activity of the native proteins. The present invention embraces counterpart proteins and polynucleotides from other species (“species orthologs”). Of particular interest are polypeptides from other mammalian species, including murine, porcine, ovine, bovine, canine, feline, equine, and other primates. Species orthologs of the human protein can be cloned using information and compositions provided by the present invention in combination with conventional cloning techniques. As used and claimed, the language “an isolated polynucleotide which encodes a polypeptide, said polynucleotide being defined by the sequences includes all allelic variants and species orthologs of this polypeptide.

The present invention also provides isolated protein polypeptides that are substantially identical to the protein polypeptides and their species orthologs. By “isolated” is meant a protein or polypeptide that is found in a condition other than its native environment, such as apart from blood and animal tissue. In a preferred form, the isolated polypeptide is substantially free of other polypeptides, particularly other polypeptides of animal origin. It is preferred to provide the poly-peptides in a highly purified form, i.e. greater than 95% pure, more preferably greater than 99% pure. The term “substantially identical” is used herein to denote polypeptides having 50%, preferably 60%, more preferably at least 80%, sequence identity to the sequence of the native proteins or species orthologs. Such polypeptides will more preferably be at least 90% identical, and most preferably 95% or more identical to the native protein, or its species orthologs. Percent sequence identity is determined by conventional methods. See, for example, Altschul et al., Bull. Math. Bio. 48: 603-616 (1986) and Henikoff and Henikoff, Proc. Natl. Acad. Sci. USA 89:10915-10919 (1992). Sequence identity of polynucleotide molecules is determined by similar methods using a ratio as disclosed above.

Variant polypeptides or substantially identical proteins and polypeptides are characterized as having one or more amino acid substitutions, deletions or additions. These changes are preferably of a minor nature, that is conservative amino acid substitutions (see Table 1) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a polyhistidine tract, protein A, Nilsson et al., EMBO J. 4:1075 (1985); Nilsson et al., Methods Enzymol. 198:3 (1991), glutathione S transferase, Smith and Johnson, Gene 67:31 (1988), or other antigenic epitope or binding domain. See, in general Ford et al., Protein Expression and Purification 2: 95-107 (1991). DNAs encoding affinity tags are available from commercial suppliers (e.g., Pharmacia Biotech, Piscataway, N.J.).

TABLE 1
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, Nmethylglycine, addo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating nonnaturally occurring amino acid residues into proteins. For example, an in vitro system can be employed wherein nonsense mutations are suppressed using chemically aminoacylated suppressor tRNAs. Other methods are described by Schultz et al. J. Org. Chem., 2003, 68, 174-176 or Science 2003, vol 299, 640. Methods for synthesizing amino acids and aminoacylating tRNA are known in the art. Essential amino acids in the polypeptides of the present invention can be identified according to procedures known in the art, such as site-directed mutagenesis or alanine scanning mutagenesis [Cunningham and Wells, Science 244: 1081-1085 (1989)]; Bass et al., Proc. Natl. Acad. Sci. USA 88:4498-4502 (1991). In the latter technique, single alanine mutations are introduced at every residue in the molecule, and the resultant mutant molecules are tested for biological activity (e.g., ligand binding and signal transduction) to identify amino acid residues that are critical to the activity of the molecule. Sites of ligand-protein interaction can also be determined by analysis of crystal structure as determined by such techniques as nuclear magnetic resonance, crystallography or photoaffinity labeling. See, for example, de Vos et al., Science 255:306-312 (1992); Smith et al., J. Mol. Biol. 224:899-904 (1992); Wlodaver et al., FEES Lett. 309:59-64 (1992). The identities of essential amino acids can also be inferred from analysis of homologies with related proteins.

Multiple amino acid substitutions can be made and tested using known methods of mutagenesis and screening, such as those disclosed by Reidhaar-Olson and Sauer, Science 241:53-57 (1988) or Bowie and Sauer Proc. Natl. Acad. Sci. USA 86:2152-2156 (1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem.30:10832-10837 (1991); Ladner et al., U.S. Pat. No. 5,223,409; Huse, WIPO Publication WO 92/06204) and region-directed mutagenesis, Derbyshire et al., Gene 46:145 (1986); Ner et al., DNA 7:127 (1988).

The present invention further provides a variety of other polypeptide fusions and related multimeric proteins comprising one or more polypeptide fusions. Polypeptide fusions can be expressed in genetically engineered cells. Auxiliary domains can be fused to the polypeptides to target them to specific cells, tissues, or macromolecules (e.g., collagen). For example, a IL-21 polypeptide or protein could be targeted to a predetermined cell type by fusing a polypeptide to a ligand that specifically binds to a receptor on the surface of the target cell. In this way, polypeptides and proteins can be targeted for therapeutic or diagnostic purposes. A IL-21 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9 (1996).

Using the methods discussed above, one of ordinary skill in the art can prepare a variety of polypeptides or allelic variants thereof and retain the properties of the wildtype protein. As expressed and claimed herein the language, “a polypeptide” includes all allelic variants and species orthologs of the polypeptide.

The protein polypeptides of the present invention, including full-length proteins, protein fragments (e.g. ligand-binding fragments), and fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multi-cellular organisms, are preferred. Techniques for manipulating cloned DNA molecules and introducing exogenous DNA into a variety of host cells are disclosed by Sambrook et al., Molecular Cloning: A Laboratory Manual, 2nd ed.(Cold Spring Harbor Laboratory Press, Cold Spring Harbor, N.Y., 1989), and Ausubel et al., ibid.

### EXAMPLES

General Procedure:

Step A:
1. Select the appropriate Caspase by determining the existence of secondary cleavage sites. This procedure may for a large part be done in silico as simple sequence analysis may exclude some candidate enzymes, however, most often only the absence of sites can be used for deciding on a particular enzyme while the presence of a site not necessarily warrants exclusion as the sequence may be unaccessible in the three-dimensional structure. In the case a potential cleavage site is present in the structure the accessibility may be probed by caspase treatment using elevated enzyme concentrations, i.e., a 1:20 or 1:50 ratio of caspase to product under conditions providing for optimal caspase activity (Stennicke & Salvesen. J.Biol.Chem. (1997) 272: 25719-25723), in order to establish the level of aberrant cleavage.
2. Generate the appropriate fusion protein by combination of the optimal linker and fusion partner/tag. TAG-Caspase site-IL21. Once decision has been made on a particular enzyme the IL21 fusion protein may be generated by genetic engineering. A very simple example of such a fusion protein may be MHHHHHH-DEVD-IL21, which would allow for purification of the product by metal chelating chromatography and processing by caspase 3 or 7 according to the aforementioned caspase specificities.
3. Express protein in a suitable expression host system. Protocols for expression of Met-IL21 into inclusion bodies (IBs) in E.coli has previously been described (WO 00/53761 or WO 04055168) and this protocol may be utilized for the fusion protein as well. Following expression the cells are harvested by centrifugation and broken open by a suitable physical or chemical technique, e.g. sonocation or French press. The IBs and cell fragments are collected by centrifugation and the pellet washed repeatedly with a suitable buffer contain detergent for solubilization of cell fragments. The semi-pure lBs are then washed with water to remove any detergent residues before they are solubilized in a suitable denaturant, typically urea or guanidine. At this point the protein may be further purified using the affinity tag according to conventional procedures.
4. Activation of desired caspase by addition of reductive equivalents. An important step in the use of thiol proteases such as the caspases is the pre-activation of the enzymes by reduction. This is most easily done by addition of DTT to the enzyme stock solution to a final concentration of 10 mM, which will result in rapid activation of the enzyme at 37° C.
5. Cleavage of folded or unfolded. Once the fusion protein has been isolated it may be cleaved directly by the caspase in buffer containing 3-4 M Urea and EDTA or reducing equivalents. Alternatively, it may be refolded using the established procedure (ref.) and the pure refolded material may then be processed in a suitable buffer. The time required to complete the processing depends on the caspase concentration, buffer composition and the N-terminal sequence of the desired IL-21 protein.
6. Separation of TAG and unprocessed material from the product. To remove released tags and unprocessed material the material is simply passed through the affinity matrix and the flow-trough containing the released IL-21 is collected. To simplify the removal of the caspases there exists several options: a) they may be produced with the same tag as IL21 which allows for removal along with unprocessed material, b) they may be covalently immobilized on beads or c) a mixed bed column containing several affinity matrixes may be used to remove unprocessed material, tags and the enzyme in one step if different tags are used.
   Pharmacological Methods

Cell pellet from fermentation of E. Coli expressing CISNN116 (NT1-C3-hIL21) was resuspended in lysis buffer containing 2 tablets/L Complete™, 200 mg/L DNAse and 20 mg/L MgCI₂. Cells were subsequently opened using a cell dispruter running at 2000 Bar. Inclusion bodies were collected by centrifugation.

The inclusion bodies (10 g) were resuspended in 100 ml 6 M Guanidinium HCl, 100 mM Tris, 40 mM Dithiothreitol (DTT) at pH 8.0. The protein was refolded using a volumetric method, by slow addition of the resuspended and reduced inclusion bodies to 2 L of 0.9 M Arginine, 10 mM NaCl, 2 mM MgCl₂, 0.5 mM KCl, 2 mM CaCl₂, 1.5 mM DTT, 4 mM L-Cystine, 50 mM Tris, pH 7.5 at 15° C.

After refoldning overnight, the protein was diluted 4 times in 25 mM Tris pH 7.5 and run on a TosoHaas sp 550c column, using a NaCl gradient as elution. Fractions containing the protein was pooled and run on a affinity column.

The buffer used for Caspase 3 proteolysis of the IL21 fusion protein was; 20 mM Tris, 0.3 M NaCl, 25 mM L-Histidine, 10 mM EDTA-Na²⁺, 1 mM GSH, pH 7.0. The pooled fractions were preheated at 37° C., prior to addition of Caspase 3. Proteolysis was run for 1 hour at 37° C. Final concentration of tag-DEVD-hIL21 and Caspase 3 were 77 μM and 90 nM, respectively.

Maldi analysis showed 87% completion of the reaction. The native hIL-21 was separated from tag-DEVD-hIL-21 fusion protein and other impurities using a Mono S column run in 25 mM Tris buffer pH 7.5 with a NaCl gradient as elution.

4 w/v % of Mannitol was added to the final fraction containing hIL-21 for stabilisation of the protein. Subsequent receptor assay (BaF assay) and DSC (differential scanning calorimetry) analysis showed that the hIL-21 produced from CISNN116 is equipotent with, and showed same melting temperature of 57° C. as previous purified batches of Met-hIL-21.

1. A method for producing a peptide comprising expressing a fusion protein comprising the peptide and an amino acid sequence recognizable by an aspartate specific protease and contacting the peptide with an aspartate specific protease to cleave the fusion protein and obtain the peptide.

2. The method of claim 1, wherein the aspartate specific protease is selected from caspases 1, 2, 3, 4, 5, 6, 7, 8, 9, and 10; Granzyme B; and CED-3

3. The method of claims 1, wherein the protease is a caspase 3.

4. The method of claim 1, wherein the peptide is a protease inhibitor.

5. The method of claim 4, wherein the protease inhibitor is aprotinin or tissue factor path-way inhibitor.

6. The method of claim 4, wherein the peptide is an insulin or an insulin precursor.

7. The method of claim 4, wherein the protein is a blood coagulation factor.

8. The method of claim 8, wherein the protein is factor VII, factor VIII, factor IX, or factor XIII.

9. The method of claim 4, wherein the peptide is a growth hormone.

10. The method of claim 4, wherein the peptide is an interleukin (IL).

11. The method of claim 10, wherein the interleukin is selected from IL-20, IL-21, IL-28, IL-29, and IL-31.

12. The method of claim 11, wherein the peptide is IL-21.

13. The method of claim 4, wherein the peptide is a glucagon like peptide-1 (GLP-1).

14. The method of claim 4, wherein the peptide is selected from glucagon, Glucagon₁₋₃₇(oxyntomodulin), glucagon-like peptide 2 (GLP-2), insulin-like growth factor-I (IGF-I), IGF-II, tissue plasminogen activator, transforming growth factor (TGF)-α, TGF-β, platelet-derived growth factor, GRF (growth hormone releasing factor), erythropoietin (EPO), protein C, exendin-3, and exentidin-4.

15. A fusion protein comprising an IL-21 and a sequence recognizable by an aspartate specific protease.

16. A fusion protein comprising a therapeutic protein portion and an aspartate specific protease recognizable sequence comprising an amino acid sequence according to Asp Glu Xaa Asp, wherein Xaa represents any amino acid residue (Formula I); Xaa₁ Val Xaa Asp, wherein Xaa₁ is Tyr, Phe, or Trp (Formula II); or Xaa₂ Glu Xaa Asp, wherein Xaa₂ is Ile, Leu, or Val (Formula III).

17. The fusion protein of claim 16, wherein the fusion protein comprises a Formula I sequence that is recognizable by caspase 2, 3, or 7, or CED-3.

18. The fusion protein of claim 16, wherein the fusion protein comprises a Formula II sequence that is recognizable by caspase 1, 4, or 5.

19. The fusion protein of claim 16, wherein the fusion protein comprises a Formula III sequence that is recognizable by caspase 6, 8, 9, 10, or Granzyme B.

20. The fusion protein of claim 16, wherein the aspartate specific protease recognizable sequence is Asp Glu Thr Asp; Asp Glu Val Asp; Asp Met Gln Asp; or Asp Gly Pro Asp.
